(19) United States
(12) Patent Application Publication
(10) Pub. No.: US 2025/0057687 A1
(43) Pub. Date: Feb. 20, 2025

(54) SLEEP APNEA RETAINER

(71) Applicant: Mark Eskander, Chadds Ford, PA (US)

(72) Inventor: Mark Eskander, Chadds Ford, PA (US)

(21) Appl. No.: 18/805,665

(22) Filed: Aug. 15, 2024

Related U.S. Application Data
(60) Provisional application No. 63/520,206, filed on Aug. 17, 2023.

(51) Int. Cl. A61F 5/56

(52) U.S. Cl. CPC A61F 5/566

(57) ABSTRACT

Exemplary embodiments are directed to a sleep apnea retainer. The sleep apnea retainer includes a top teeth section including cavities configured to at least partially receive a top row of teeth of an individual. The sleep apnea retainer includes a bottom teeth section including cavities configured to at least partially receive a bottom row of teeth of an individual. The top teeth section is connected to the bottom teeth section at a distal end of the sleep apnea retainer to form a single body.

This application claims the benefit of a co-pending U.S. Provisional Patent Application No. 63/520,206, which was filed on Aug. 17, 2024. The entire content of the foregoing provisional application is incorporated herein by reference.

### TECHNICAL FIELD

The present disclosure relates to a sleep apnea retainer and, in particular, to a sleep apnea retainer configured to maintain a natural anatomic position of teeth of an individual during sleeping.

### BACKGROUND

Sleep apnea is a sleep disorder which pauses breathing or creates moments of shallow breathing during sleep during obstructions of the airway. An individual's normal anatomical position of the teeth when standing typically positions the top teeth slightly forward of the bottom teeth at the proximal end. However, when the individual lies down to sleep, the top teeth and jaw can move backwards to align the teeth and obstruct the passage of air, resulting in sleep apnea.

Thus, a need exists for a sleep apnea retainer that maintains the position of the top and bottom teeth in an optimal, rigid relationship to prevent sleep apnea. These and other needs are addressed by the sleep apnea retainer of the present disclosure.

### SUMMARY

In accordance with embodiments of the present disclosure, an exemplary sleep apnea retainer is provided. The sleep apnea retainer includes a top teeth section including cavities configured to at least partially receive a top row of teeth of an individual. The sleep apnea retainer includes a bottom teeth section including cavities configured to at least partially receive a bottom row of teeth of an individual. The top teeth section is connected to the bottom teeth section at a distal end of the sleep apnea retainer to form a single body.

The top teeth section is rigidly connected to the bottom teeth section to prevent movement between the top and bottom teeth sections. A proximal end of the top teeth section is misaligned with a proximal end of the bottom teeth section by an offset distance. The proximal end of the top teeth section is positioned in front of the proximal end of the bottom teeth section.

In some embodiments, the top teeth section and the bottom teeth section can be formed from a silicone material. In some embodiments, the top teeth section and the bottom teeth section can be formed from a plastic material. The top teeth section is connected to the bottom teeth section at two opposing connecting sections at the distal end of the sleep apnea retainer. The two opposing connecting sections are integrally formed with the top and bottom teeth sections. The retainer can include an opening between the top and bottom teeth sections. The opening is configured for passage of air through the sleep apnea retainer.

In accordance with embodiments of the present disclosure, an exemplary sleep apnea retainer is provided. The sleep apnea retainer includes a base section including a top section and a bottom section. The top section is connected to the bottom section at a distal end of the base section to form a single body. The sleep apnea retainer includes a top teeth section including cavities configured to at least partially receive a top row of teeth of an individual. The sleep apnea retainer includes a bottom teeth section including cavities configured to at least partially receive a bottom row of teeth of an individual. The top teeth section is configured to be engaged with the top section of the base section, and the bottom teeth section is configured to be engaged with the bottom section of the base section.

The base section can be fabricated from a different material than the top and bottom teeth sections. In some embodiments, the base section can be fabricated from a three-dimensional printed orthodontic plastic, and the top and bottom sections are fabricated from silicone. The top and bottom teeth sections can be custom-molded to be complementary to teeth of an individual.

The top section is rigidly connected to the bottom section to prevent movement between the top and bottom sections. A proximal end of the top section is misaligned with a proximal end of the bottom section by an offset distance such that the top teeth section is misaligned by the offset distance relative to the bottom teeth section. The proximal end of the top teeth section is positioned in front of the proximal end of the bottom teeth section. The top section is connected to the bottom section at two opposing connecting sections at the distal end of the sleep apnea retainer. The two opposing connecting sections are integrally formed with the top and bottom sections. The sleep apnea retainer includes an opening between the top and bottom sections. The opening is configured for passage of air through the sleep apnea retainer.

Other objects and features will become apparent from the following detailed description considered in conjunction with the accompanying drawings. It is to be understood, however, that the drawings are designed as an illustration only and not as a definition of the limits of the invention.

### BRIEF DESCRIPTION OF THE DRAWINGS

To assist those of skill in the art in making and using the disclosed sleep apnea retainer, reference is made to the accompanying figures, wherein:

FIG. 1 is a side view of a sleep apnea retainer according to the present disclosure;

FIG. 2 is a rear view of a sleep apnea retainer of FIG. 1;

FIG. 3 is a top view of a sleep apnea retainer of FIG. 1; and

FIG. 4 is a bottom view of a sleep apnea retainer of FIG. 1.

### DESCRIPTION OF EXEMPLARY EMBODIMENTS

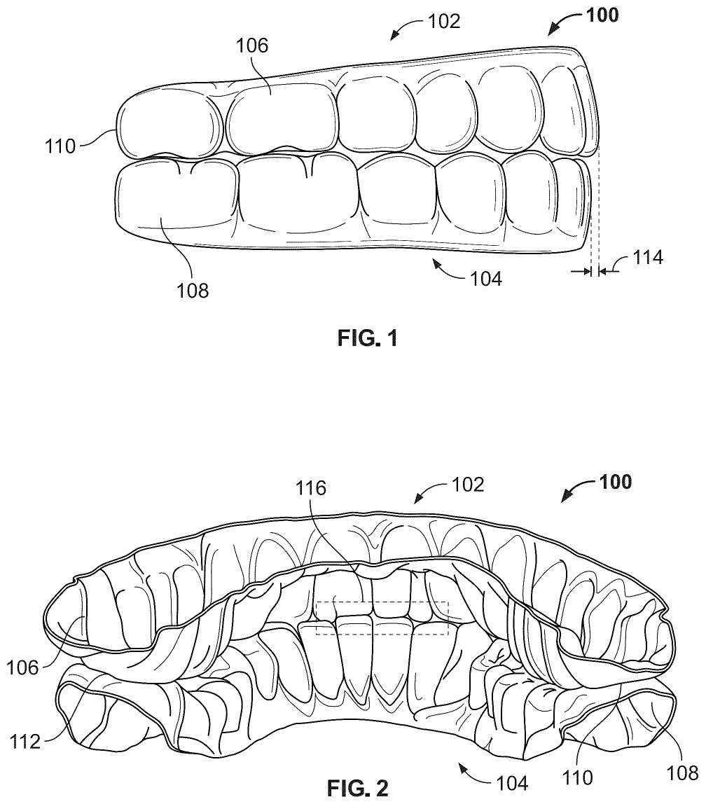

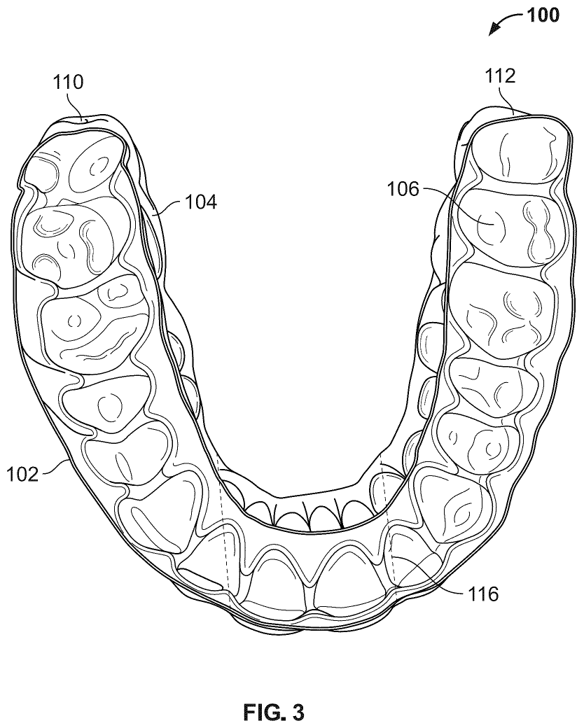

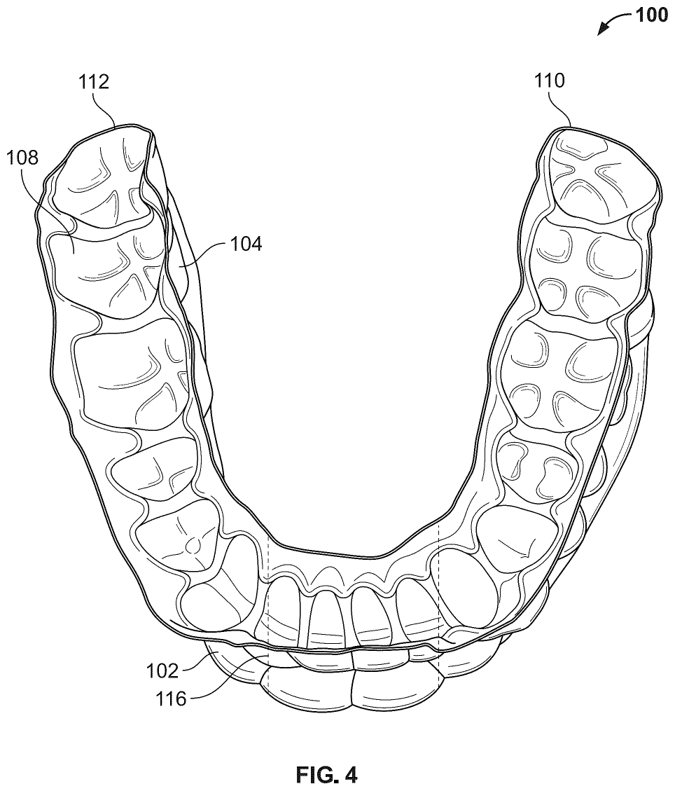

FIGS. 1-4 are side, rear, top and bottom views of an exemplary sleep apnea retainer 100 (hereinafter referred to as “retainer 100”). The retainer 100 is configured to be worn at night to prevent snoring and sleep apnea of the individual. As discussed herein, the retainer 100 ensures that the top teeth are shifted slightly in front of the bottom teeth when lying down for sleep, thereby keeping the natural misalignment (or offset alignment) of the teeth when the individual is upright. The natural alignment from the upright position is thereby maintained in the horizontal or lying down position to ensure proper reduction or prevention of sleep apnea. The top and bottom sections of the retainer 100 are connected at opposing ends to define a single-piece design. The one-piece or single-piece design ensures proper alignment of the teeth and accuracy in design.

The retainer 100 includes a top teeth section 102 and a bottom teeth section 104. The top teeth section 102 includes cavities 106 molded in a customized manner to the top teeth of the individual, and the bottom teeth section 104 includes cavities 108 molded in a customized manner to the bottom teeth of the individual. The cavities 106, 108 are configured and dimensioned to at least partially receive the respective teeth of the individual. In particular, the cavities 106, 108 are configured to receive the uppermost part of the respective teeth, as well as at least some portions of the sides of the teeth. In some embodiments, the entire retainer 100 can be formed from a silicone and/or plastic material, with this material in the cavities 106, 108 providing friction to releasably maintain the position of the teeth of the individual.

In some embodiments, the retainer 100 can be formed as a three-part structure, including a single-piece base formed from a hard plastic material (e.g., three-dimensional printed orthodontic plastic, or the like) that includes the base for both the top and bottom sets of teeth, and is connected on opposing sides. Such retainer further includes softer “retainer” components molded from the silicone and/or plastic material to perfectly match the position of the individual's jaw and/or teeth to avoid misalignment. The base structure can therefore be used for any individual, while the top and bottom teeth sections are customized to the individual and attachable to the base structure. In some embodiments, the base structure can include openings and/or extensions configured to engage with complementary openings and/or extensions of the softer material of the top and bottom teeth sections to allow engagement assembly of the components together.

With reference again to FIGS. 1-4, the distal end of the retainer 100 defines the frontmost teeth of both the top and bottom teeth of the individual, while the proximal end of the retainer 100 defines the innermost teeth of both the top and bottom teeth of the individual. The proximal end of the retainer 100 includes two connecting sections 110, 112 on the respective right and left sides of the retainer 100. The connecting sections 110, 112 are integrally formed with the top and bottom teeth sections 102, 104 such that the retainer 100 defines a single structure (or at least a single based structure). The connecting section 110, 112 create a rigid connection between the top and bottom teeth sections 102, 104, preventing any separation of the top and bottom teeth sections 102, 104. The single structure of the retainer 100 ensures that proper alignment of the top and bottom teeth is maintained.

In particular, the natural alignment of the teeth when the individual is upright is with a slightly forward shift of the top teeth relative to the bottom teeth, as represented by the offset distance 114 in FIG. 1. The offset distance 114 can be customized and selected to match or substantially match the upright anatomy of the individual to protect against occlusion of the airway. Typically, when the individual lies down to sleep, this natural forward shift of the top teeth shifts rearward due to gravity and jaw movement, resulting in snoring and/or sleep apnea. The single structure of the retainer 100 with the offset distance 114 ensures that even in the lying down position, the forward shift of the top teeth remains, creating a natural anatomic position (e.g., an optimal anatomic position) for the individual when lying down. By maintaining the natural anatomic position of the top and bottom teeth relative to each other, the retainer 100 prevents movement of teeth, reduces the risk for orthodontic problems (such as recession and gum disease), and prevents temporomandibular joint dysfunction (TJD). In some embodiments, the retainer 100 can include an opening 116 formed between the top and bottom teeth sections 102, 104 to allow for air passage through the retainer 100 during use. The opening 116 results in comfortable breathing of the individual while wearing the retainer 100.

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

1. A sleep apnea retainer, comprising:
a top teeth section including cavities configured to at least partially receive a top row of teeth of an individual; and
a bottom teeth section including cavities configured to at least partially receive a bottom row of teeth of an individual;
wherein the top teeth section is connected to the bottom teeth section at a distal end of the sleep apnea retainer to form a single body.

2. The sleep apnea retainer of claim 1, wherein the top teeth section is rigidly connected to the bottom teeth section to prevent movement between the top and bottom teeth sections.

3. The sleep apnea retainer of claim 1, wherein a proximal end of the top teeth section is misaligned with a proximal end of the bottom teeth section by an offset distance.

4. The sleep apnea retainer of claim 3, wherein the proximal end of the top teeth section is positioned in front of the proximal end of the bottom teeth section.

5. The sleep apnea retainer of claim 1, wherein the top teeth section and the bottom teeth section is formed from a silicone material.

6. The sleep apnea retainer of claim 1, wherein the top teeth section and the bottom teeth section is formed from a plastic material.

7. The sleep apnea retainer of claim 1, wherein the top teeth section is connected to the bottom teeth section at two opposing connecting sections at the distal end of the sleep apnea retainer.

8. The sleep apnea retainer of claim 7, wherein the two opposing connecting sections are integrally formed with the top and bottom teeth sections.

9. The sleep apnea retainer of claim 1, comprising an opening between the top and bottom teeth sections.

10. The sleep apnea retainer of claim 9, wherein the opening is configured for passage of air through the sleep apnea retainer.

11. A sleep apnea retainer, comprising:
a base section including a top section and a bottom section, wherein the top section is connected to the bottom section at a distal end of the base section to form a single body;
a top teeth section including cavities configured to at least partially receive a top row of teeth of an individual; and
a bottom teeth section including cavities configured to at least partially receive a bottom row of teeth of an individual;
wherein the top teeth section is configured to be engaged with the top section of the base section, and the bottom teeth section is configured to be engaged with the bottom section of the base section.

12. The sleep apnea retainer of claim 11, wherein the base section is fabricated from a different material than the top and bottom teeth sections.

13. The sleep apnea retainer of claim 12, wherein the base section is fabricated from a three-dimensional printed orthodontic plastic, and the top and bottom sections are fabricated from silicone.

14. The sleep apnea retainer of claim 11, wherein the top and bottom teeth sections are custom-molded to be complementary to teeth of an individual.

15. The sleep apnea retainer of claim 11, wherein the top section is rigidly connected to the bottom section to prevent movement between the top and bottom sections.

16. The sleep apnea retainer of claim 1, wherein a proximal end of the top section is misaligned with a proximal end of the bottom section by an offset distance such that the top teeth section is misaligned by the offset distance relative to the bottom teeth section.

17. The sleep apnea retainer of claim 16, wherein the proximal end of the top teeth section is positioned in front of the proximal end of the bottom teeth section.

18. The sleep apnea retainer of claim 11, wherein the top section is connected to the bottom section at two opposing connecting sections at the distal end of the sleep apnea retainer.

19. The sleep apnea retainer of claim 18, wherein the two opposing connecting sections are integrally formed with the top and bottom sections.

20. The sleep apnea retainer of claim 11, comprising an opening between the top and bottom sections, wherein the opening is configured for passage of air through the sleep apnea retainer.
